(19) United States
(12) Patent Application Publication
(10) Pub. No.: US 2019/0117883 A1
(43) Pub. Date: Apr. 25, 2019

(54) SYRINGE FOR DELIVERING MEDICATIONS TO IMPLANTED DRUG DELIVERY SYSTEMS AND METHODS FOR USE THEREOF

(71) Applicant: Cerebral Therapetuics LLC, Aurora, CO (US)

(72) Inventors: Daniel J. Abrams, Denver, CO (US); Michael S. Canney, Denver, CO (US)

(73) Assignee: Cerebral Therapetuics LLC, Aurora, CO (US)

(21) Appl. No.: 16/168,445

(22) Filed: Oct. 23, 2018

Related U.S. Application Data
(60) Provisional application No. 62/575,767, filed on Oct. 23, 2017.

(51) Int. Cl. A61M 5/142; A61M 39/02

(52) U.S. Cl. CPC A61M 5/14276; A61M 39/0208; A61M 2039/0232; A61M 2209/045; A61M 2005/14284

(57) ABSTRACT

The invention provides for ensuring proper transfer of a fluid to a medical device using a syringe by determining a position of a distal end of the needle relative to the medical device and/or a subject using a sensor. If the position of the distal end of the needle relative to a desired point of injection on a medical device and/or the subject is determined to be within predefined parameters transfer of a content of the syringe to the medical device takes place or may be inhibited by a lockout system.

### FIELD OF THE INVENTION

The present disclosure generally relates to direct brain administration of a medicant, and includes a method of treatment and a device for use with an implantable device for the delivery of medication or other fluids to a patient.

### BACKGROUND OF THE INVENTION

The delivery of medications directly to specific parts of the body has several advantages over systemic administration. Lower doses of medication can be used with site specific administration and systemic toxicities of drug therapies can be mitigated. Furthermore, delivery port implantable devices are used regularly in the delivery of chemotherapy and medications to allow direct administration of certain medications best administered through direct access to the venous system, central nervous system and peritoneal cavity.

In some organs of the body, direct administration of certain drugs may be required to achieve therapeutic doses of medication without inducing serious toxicity. For example, the blood-brain barrier (BBB) protects the brain from potentially toxic substances, but also restricts the passage of most drug molecules. Because of the BBB, direct administration of drugs may be required to achieve therapeutic concentrations for the treatment of cancer, neurodegenerative diseases such as Alzheimer's (AD) and Parkinson's Disease (PD), and other conditions.

Alternatively, devices for local, repeatable, and chronic drug delivery can be used. These devices contain catheters that distribute drug to specific tissues and may or may not contain a reservoir for storing drug. These devices include implantable drug pump systems, Ommaya/Rickham reservoirs, and Port-A-Cath® devices. These devices deliver either a bolus or slow infusion of drugs to specific regions of the body. The types of drugs that can be used range from pain medications (e.g., morphine) to chemotherapeutics (e.g., methotrexate, cytarabine).

Examples of implantable drug pump systems include the Flowonix Prometra II and the Medtronic Synchromed devices. These implantable pumps are programmable, refillable, and are used to chronically deliver drugs to a patient. These pumps may contain drug reservoirs with an internal volume of from about 10 to 40 mL that are refilled regularly by patient caregivers. These devices are currently used for the delivery of pain medications to the spinal region of patients, as well as for the local delivery of chemotherapy. To refill these devices, a needle (−22 gauge) is used to pierce a septum underneath the skin that is located in the center of the pump, after which drug is injected into the internal pump reservoir to refill it.

Another type of local drug delivery device is an Ommaya/Rickham style reservoir. These reservoirs are used to locally deliver medications, typically to the cerebrospinal fluid. These devices can be used to deliver a bolus via a syringe or a continuous infusion of drug by using an external syringe pump. These medications can include drug therapies such as chemotherapy drugs (e.g. methotrexate) for patients with primary brain tumors or leptomeningeal metastases.

Another type of device is an implantable port for repeated delivery of drug therapies. These devices include the Eco Port, Clip-a-Port, SmartPort, Microport, Bardport, PowerPort, Passport, Port-a-Cath, Medi-Port, and Bioflo. These devices have a port with a septum underneath the skin. A catheter runs from the port to a catheter that is surgically inserted into a vein. These devices have a wide variety of uses including delivery of chemotherapy, delivery of coagulation factors in patients with severe hemophilia, delivery of antibiotics, or delivery of pain medications. As stated above, various devices exist for repeated or chronic delivery of medication directly to specific organs/regions of the body.

All of the above implantable drug delivery devices have access ports underneath the skin that must be located and accessed with a needle to refill them. Locating these ports and accessing them can be problematic and there is currently no feedback system to ensure that the refill needle is properly placed in the port before the clinician injects a medication. Improper delivery of medication to regions around the implanted port can cause severe reactions or even be fatal to a patient. For example, there have been several reports of accidental injection of drugs into the wrong port of implanted pumps that have resulted in death. Typically, implantable pumps have two ports, one for refill and a second port for direct access to the catheter. If drug is injected into the second port, it can be delivered in very high concentrations directly to the CSF or other regions in the body and be lethal for the patient.

Because of these issues, methods of detecting proper needle penetration into the septums/reservoirs of the ports of implanted pumps have been proposed. In U.S. Publication No. US20140228765A1 to Burke et aL (“Burke”) entitled “Needle Penetration Detection Method and Device for Refillable and Implantable Drug Delivery Systems”, a method is described for detecting proper needle insertion into an implanted pump. The detection mechanism is located within the implantable pump. Several methods are proposed to detect that the needle fully penetrates the septum and is located in the reservoir-side of the refill port. The proposed methods include infrared (IR) illumination, LED optical emitters, acoustic wave emitters, fluorescent materials, measurement of electrical impedance within the pump, magnetic or inductive sensors. Burke also proposes the integration of a magnetic-field sensor within the needle tip that provides feedback of the position of the needle within the pump. A further method that is proposed is to integrate a mechanical switch at the bottom of the refill port that activates when the needle strikes it A further method involves sensing the internal pressure of the pump as the needle penetrates the reservoir or as it is refilled. For the various methods proposed that involve detection systems within the pump, the signals generated within the pump must be wirelessly communicated to the refill system. Alternatively, a magnetic-field sensor system is proposed to be integrated into the refill wand and requires that a magnetic be integrated into the pump/septum region.

Burke describes a needle penetration detector in an implantable pump that uses an induction coil around the septum. By using an inductance coil in the region around the septum, the pump can determine whether the needle is properly inserted into the pump.

While the above described methods are proposed for determining proper insertion of the refill tool into the implanted pump, all of the proposed methods require modifications to the implanted pump and cannot be used with multiple devices. Hundreds of thousands of patients have been implanted with pumps and all of the proposed methods cannot be used with these patients who have existing devices. These pumps are implanted and will operate for 5-10 years without the need for replacement. Integrating new features into devices that are already approved is also costly and requires significant regulatory work and verification before such modifications can be made available in a commercially available medical device. Furthermore, if the proposed devices malfunction, the entire pump device must be replaced, requiring invasive surgery.

It would be desirable, therefore, to ensure that refill of implanted drug pump devices and access ports can be performed using a device that does not require alteration of existing devices and which actively prevents the user from refilling device if the needle tip is not properly inserted in the device during the refill procedure.

### SUMMARY

A device, system and method for ensuring proper transfer of a fluid out of a syringe is provided. The method includes inserting a distal end of a needle of a syringe into a target area for delivery of a fluid inside the syringe. The needle includes sensors along its length which sense a position of a distal end of the needle and transfers sensed information back to a microprocessor. The microprocessor processes the information and conveys it to an electronic display making it possible to determine the position of the distal end of the needle is within a predefined distance relative to a desired injection point of the target area. When the display indicates the distal end of the needle is correctly positioned, the microprocessor allows for the opening of a valve making it possible to transfer the fluid out but preventing transfer out of the fluid when the distal end of the needle is not properly positioned. Although the sensors are in physical and electronic connection with the needle and with the microprocessor, it is possible for the microprocessor to be separate from the syringe along with the electronic display.

Proper transfer of a fluid to a medical device using a syringe is carried out by inserting a distal end of a needle of a syringe into a medical device after determining a position of a distal end of the needle relative to the medical device and/or a subject is within predefined parameters using an electronic and/or physical sensor.

In some embodiments, the method may include inhibiting transfer of a content of the syringe to the medical device if the position of the distal end of the needle relative to the medical device and/or the subject is not within the predefined parameters. The content may be inhibited by a lockout system.

In some embodiments, the method may include continuously determining, during use, if the position of the distal end of the needle relative to the medical device and/or the subject is within predefined parameters. The method may include inhibiting transfer of a content of the syringe to the medical device if the position of the distal end of the needle relative to the medical device and/or the subject is not within the predefined parameters. The method may include resuming transfer of the content of the syringe to the medical device if the position of the distal end of the needle relative to the medical device and/or the subject is within the predefined parameters. The method may include reducing a functional aspect of the content of the syringe using an anti-tamper mechanism.

In some embodiments, the medical device may include a container wherein the content of the syringe are transferrable to during use.

In some embodiments, the method may include determining a position of a distal end of the needle by determining a resistance to a current applied to at least a portion of the needle.

The method may include determining a resistance to a current applied to at least a portion of the needle using one or more electrodes associated with the syringe. The one or more electrodes may include three sets of electrodes. The three sets of electrodes may include a first set of electrodes positionable in a subject's tissue, a second set of electrodes positionable in a container of the medical device, and a third set of electrodes positionable in a septum of the medical device.

In some embodiments, the method may include determining a resistance to a current applied to at least a portion of the needle using at least two sets of electrodes associated with the syringe. A first substance may be identified using a first set of electrodes based on a first determined resistance. A second substance may be identified using a second set of electrodes based on a second determined resistance.

In some embodiments, the medical device may include an implanted device at least partially positioned in the subject. The method may include sending and/or receiving at least one packet of electronic information to and/or from the implanted device using an electronic communications system associated with the syringe or using one or more electrodes associated with the syringe.

In some embodiments, the method may include determining a position of a distal end of the needle by emitting short ultrasonic pulses from at least a portion of the needle. The pulses may be emitted by using an ultrasonic transducer associated with the syringe. An ultrasonic transducer may be used to determine a relative amount of material in a container of the medical device.

In some embodiments, the syringe may include at least one electrode and at least one ultrasonic transducer associated with the syringe.

An aspect of the invention includes a syringe system comprised of a tubular body open at its proximal end and opening to a hallow needle at its distal end. An electrical sensor or series of sensors are positioned along the length of the needle. A signal detector receives an interprets signals from the sensor.

In an aspect of the invention, sensors are electrodes and the device further includes a power source in connection with the sensor and the signal detector may be an ammeter.

In an aspect of the invention, the syringe system may be comprised of a first set, second set, and third set of electrodes which are spaced along the needle. Multiple sets of electrodes may be used as needed with the electrodes detecting the position of the needle and sending signals back to the signal detector.

Another aspect of the invention includes a syringe system for use in insuring proper transfer of a fluid to a medical device or to a patient using a syringe. The use includes inserting the distal end of the needle into a medical device or a patient and determining the position of the distal end of the needle relative to a receiving area in either the medical device or the patient by use of one or more sensors positioned on the needle. The position of the needle is determined by interpreting the signals received from the sensors indicating that the distal end of the needle is in a preferred position or a predefined parameter is met after which fluid is forced through the needle into the receiving area.

In an aspect of the invention, the sensors send information including packets of electronic information to a receiving device which interprets the information in terms of where the distal end of the needle is positioned resulting in an indication being given that the distal end of the needle is correctly positioned for the user to dispense fluid through the needle to a medical device or patient.

An aspect of the invention includes the use of a syringe or syringe system in a method for insuring proper transfer of a fluid to a medical device or patient using the syringe wherein the method includes inserting a distal end of the needle into the medical device for a patient, determining the position of the distal end of the needle relative to a fluid receiving area of the medical device or patient using the sensor and determining the position of the distal end of the needle relative to the medical device as being within predetermined parameters relative to the receiving area in the medical device or patient.

These and other objects, advantages, and features of the invention will become apparent to those persons skilled in the art upon reading the details of the device, system and method as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 depicts an embodiment of a smart needle device and pre-filled syringe being used to refill an implanted drug pump placed in the abdomen of a patient.

FIG. 2 depicts an embodiment of the proposed smart needle system attached to a syringe. The smart needle system contains a battery, microprocessor, and valve.

FIG. 3 depicts an embodiment of the proposed smart needle system connected to a separate system containing the electronics to drive the electrical contacts on the smart needle.

FIG. 4 depicts an embodiment of the proposed smart needle system inserted into an implantable pump.

FIG. 5 depicts an embodiment of a smart needle device used with an external infusion pump and an ommaya reservoir electrically coupled wirelessly.

FIG. 6 depicts an embodiment of a smart needle device used with an external infusion pump and an ommaya reservoir.

FIG. 7 depicts an embodiment of a representation of a smart acoustic syringe

FIG. 8 depicts an embodiment of a representation of a smart acoustic syringe.

FIG. 9 depicts an embodiment of a detail of the needle tip of the smart acoustic needle.

FIG. 10 depicts an embodiment of a flow chart of the method of use of the smart needle system with an implantable pump.

FIG. 11 depicts an embodiment of a flow chart of the method of use of the smart needle system with an external infusion pump.

FIG. 12 depicts an embodiment of a flow chart of the method of use of the smart needle system with an ultrasonic transducer at the tip.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third die electrically connected to the module substrate” does not preclude scenarios in which a “fourth die electrically connected to the module substrate” is connected prior to the third die, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

The scope of the present disclosure includes any feature or combination of features disclosed herein (either explicitly or implicitly), or any generalization thereof, whether or not it any or all of the problems addressed herein. Accordingly, new claims may be formulated during prosecution of this application (or an application claiming priority thereto) to any such combination of features. In particular with reference to the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for the purpose of describing particular embodiments only and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DETAILED DESCRIPTION OF THE INVENTION

Before the present device, system and methodology are described, it is to be understood that this invention is not limited to particular embodiment described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a needle” includes a plurality of such needles and reference to “the predefined parameter” includes reference to one or more parameters and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Unless defined otherwise, all technical and scientific terms used herein have the same meaning as commonly understood by one of ordinary skill in the art.

The term “catheter” as used herein generally refers to medical devices that can be inserted in the body to treat diseases or perform a surgical procedure.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

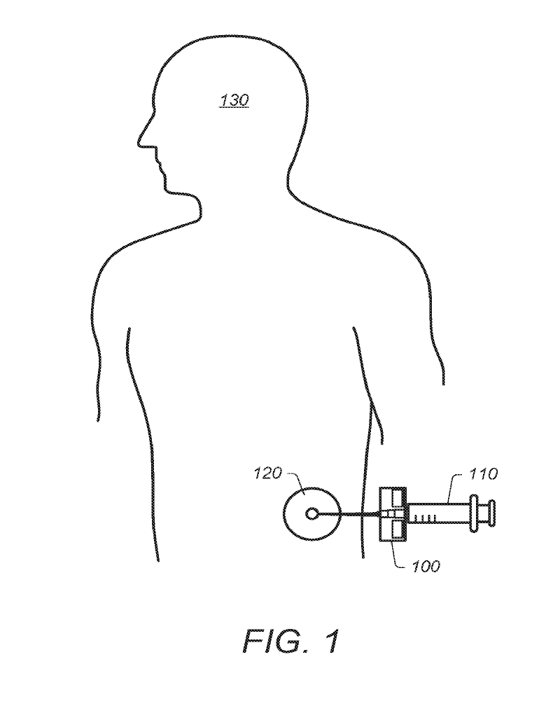

The present disclosure relates to a smart needle device that can sense when it is properly positioned into an implanted access port in a pump or port using a sensor that is integrated into the refill tool. FIG. 1 depicts an embodiment of a smart needle device 100 and pre-filled syringe 110 being used to refill an implanted drug pump 120 placed in the abdomen of a patient 130. The implanted drug pump can be connected to a catheter line to deliver drugs to the spinal region, the brain, or other regions in the body. In some embodiments, the smart needle device includes a lockout system to prevent injection of medication unless the refill needle is properly positioned in the access port. In some embodiments, the smart needle device may be used in combination with an existing syringe.

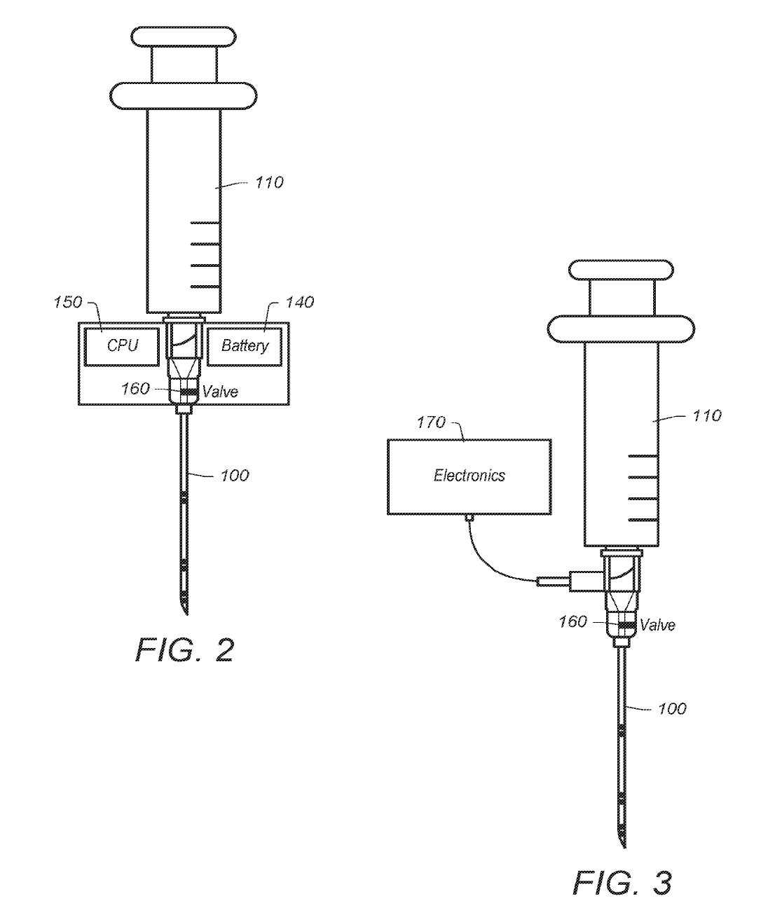

In some embodiments, the smart needle device may be used as part of a pre-filled syringe. In some embodiments, the smart needle device may be used with an external infusion pump. The smart needle device may include a single-use device or may be reusable (e.g., may be sterilized using standards known in the industry) and used for multiple refills with different syringes. A lockout device may prevent medication injection. The lockout device may continuously detect that the needle is properly placed into the implanted device/port during medication injection. This feature may be especially beneficial for long and slow infusions, such as when the needle device must be placed in the port for tens of minutes to several hours. The smart needle device can thus block medication injection if the needle is displaced or moved during infusion. FIG. 2 depicts an embodiment of the proposed smart needle system 100 attached to a syringe 110. The smart needle system 100 may include a battery 140, microprocessor 150, and valve 160. The system may be able to deliver low voltage signals to the electrodes on the needle tip and to measure the electrical impedance. By the electrical impedance, the microprocessor can open the micro valve and the practitioner can refill the implanted device. FIG. 3 depicts an embodiment of the proposed smart needle system 100 connected to a separate system 170 containing the electronics to drive the electrical contacts on the smart needle. All of these features, which are beneficial and substantially differentiate from that described previously, are described in detail in the text and figures herein.

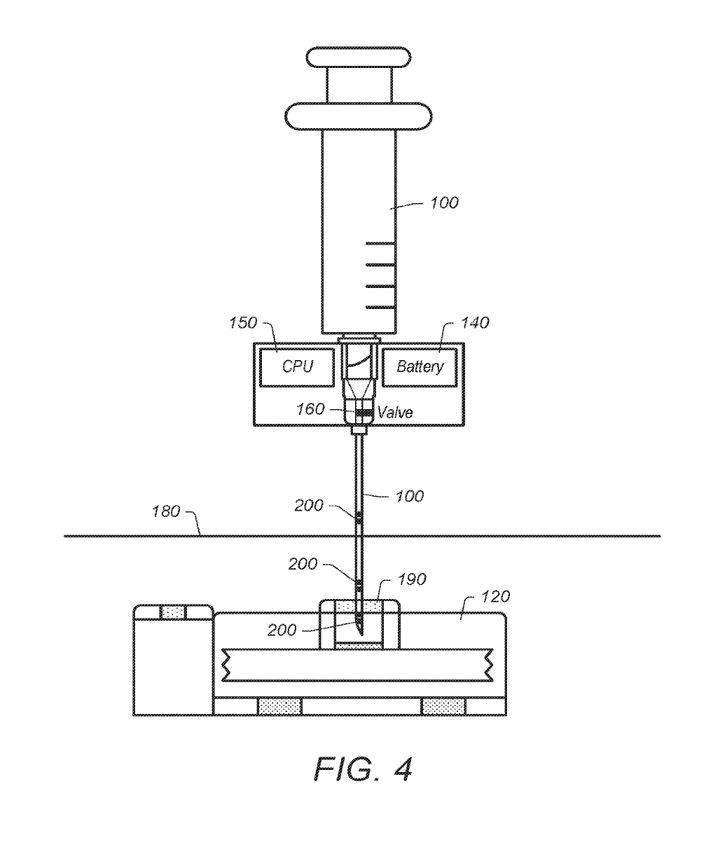

In one embodiment of the device, a needle with electrodes on the outside allows for sensing of the position of the syringe as it is being inserted into an implanted drug port. FIG. 4 depicts an embodiment of the proposed smart needle system 100 inserted into an implantable pump 120. The needle first penetrates the skin and up to several centimeters of body tissue 180 before entering a septum 190 in the center of the implanted pump. Once properly inserted through the septum, the electrodes 200 on the needle tip can sense that they are properly placed within the device and can allow for refill of the device. To operate the device, each pair of electrodes may be connected to a small continuous voltage signal that is safe for the patient (e.g. <1 V).

By monitoring the current and calculating the impedance when the voltage signal is applied, the system can calculate the resistance. Low values of resistance correspond to the needle's initial insertion into the body (which is conductive), the insertion through the septum (where both electrodes are insulated and no current will pass, thus the resistance will be very high), and finally, the placement of the needle in a proper orientation inside the drug reservoir.

The voltage signal can be either a continuous low voltage signal or may be a time-varying voltage signal such as a frequency-dependent voltage signal so that a complex impedance is calculated at varying frequency components. FIG. 5 depicts an embodiment of a smart needle device 100 used with an external infusion pump 210 and a reservoir 220 (e.g., an ommaya reservoir) electrically coupled wirelessly. The smart needle device detects when it is properly placed in the reservoir of the subject 130 and opens up the microvalve to allow drug to pass from the external infusion pump to the reservoir. The smart needle may further communicate with the external infusion pump through RFID or other means to notify the pump that infusion of drug can begin.

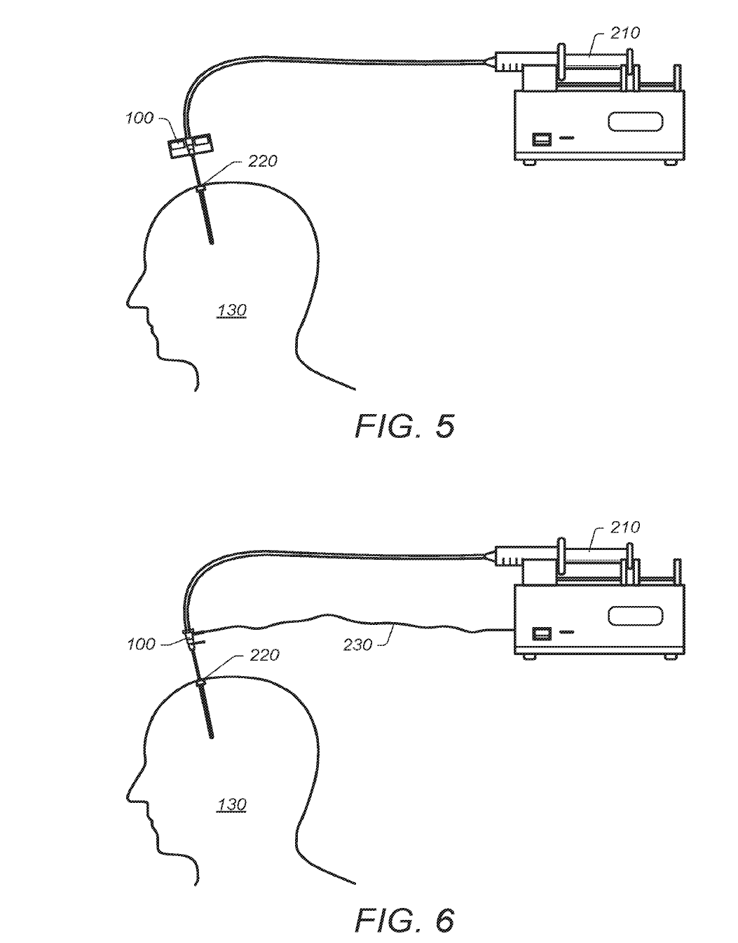

FIG. 6 depicts an embodiment of a smart needle device 100 used with an external infusion pump 210 and a reservoir 220. The smart needle device detects when it is properly placed in the reservoir and opens up the microvalve to allow drug to pass from the external infusion pump to the ommaya. The smart needle may be driven by electronics contained in the external infusion pump. The needle connects to the external pump through a separate electrical cable 230.

The needle may have, for example, three different sets of electrodes on the needle tip so that when the needle is properly placed, one set is in the tissues of the body, one set is in the septum, and the third set is in the drug reservoir. By validating that each of these electrodes has an impedance within a specified range, the user can be certain that the needle is properly positioned. Furthermore, the electrodes at the needle tip, which are inserted into the drug reservoir, may measure if the reservoir is full or empty and could further monitor the level of drug.

In some embodiments, small electrodes are formed on the tip of a needle in various ways. For an implantable pump system, a 22 gauge needle is typically used for refill of the device. Electrodes, for example in gold or silver, may be patterned on the outside of the needle by using various materials and techniques that are common in the semiconductor industry. Furthermore, since very low voltages will be used, the insulating materials may be very thin (-microns). Thus, by using alternating layers of biocompatible insulating materials such as parylene and copper/gold electrical tracks, multiple electrical contacts may be patterned on the outside of the needle connection device. The tracks used for the electrical transmission of the voltage signals may be very thin (<100 urn) since low voltage signals are used. The outer circumference of a 22 gauge needle is 2 mm, giving space for 10 or more potential tracks for electrical signals on the outer circumference. In some embodiments, electrodes may be formed using alternating insulating/conducting tubes on the interior of the needle.

In some embodiments, electrodes on the needle may be used to send information to the implanted device. For example, in the case of opiates, the smart needle/syringe may send information to the pump that corresponds to the drug and that can provide for tracking of the medication to ensure that it was properly used for refill of the pump instead of diverted for uses. The interface between the syringe and needle contains electronics that can send low voltage signals to the electrical contacts and that can process them with a microprocessor or the electronics in the hub can be passive and the power supply and other electronics may be located in an external base unit.

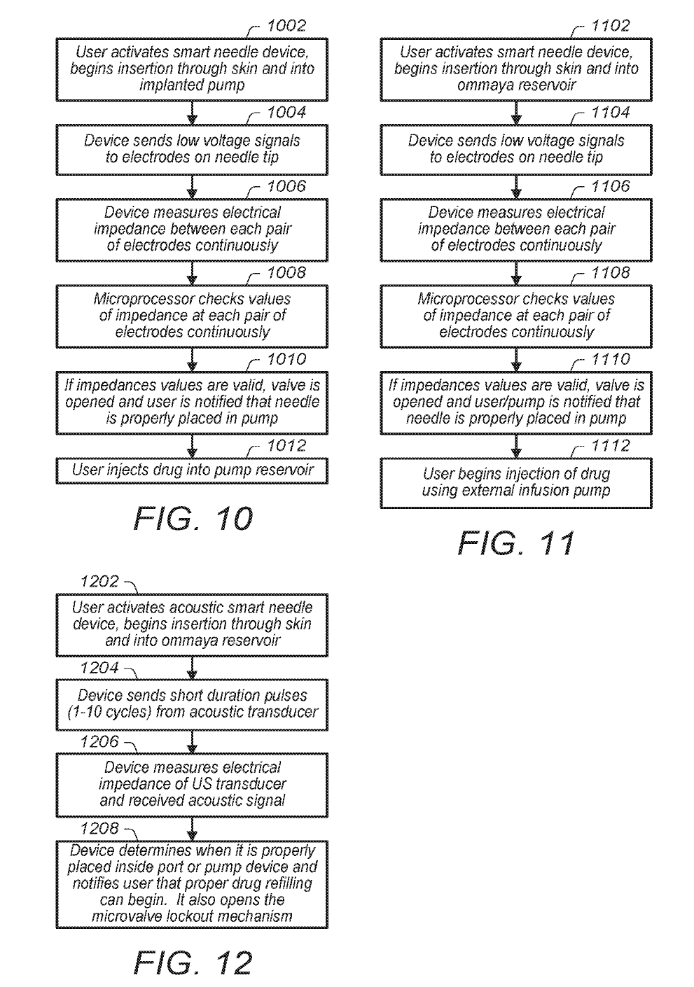

FIG. 10 depicts an embodiment of the method of use of the smart needle system 100 with an implantable pump 120. The user activates the smart needle device prior to insertion in the patient (1002). Activation of the device begins stimulation of the electrical contacts (1004) on the needle tip and measurements of the electrical impedances (1006), which are processed by the microprocessor (1008). When the correct values of impedances are measured, the system activates the micro valve at the needle tip (1010) to allow the user to inject the drug into the device (1012).

FIG. 11 depicts an embodiment of the method of use of the smart needle system 100 with an external infusion pump 210. The user activates (1102) the smart needle device prior to insertion in the patient. Activation of the device begins stimulation of the electrical contacts on the needle tip (1104) and measurements of the electrical impedances (1106), which are processed by the microprocessor (1108). When the correct values of impedances are measured, the system activates the micro valve at the needle tip (1110) to allow the user to inject the drug into the device (1112). The smart needle may further communicate with the external infusion pump through RFID, direct electrical connection, or other means to notify the pump that injection of drug can proceed.

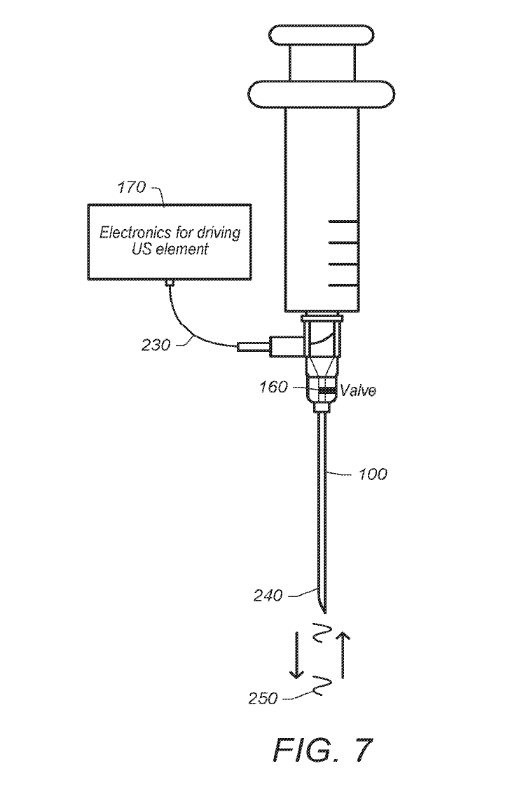

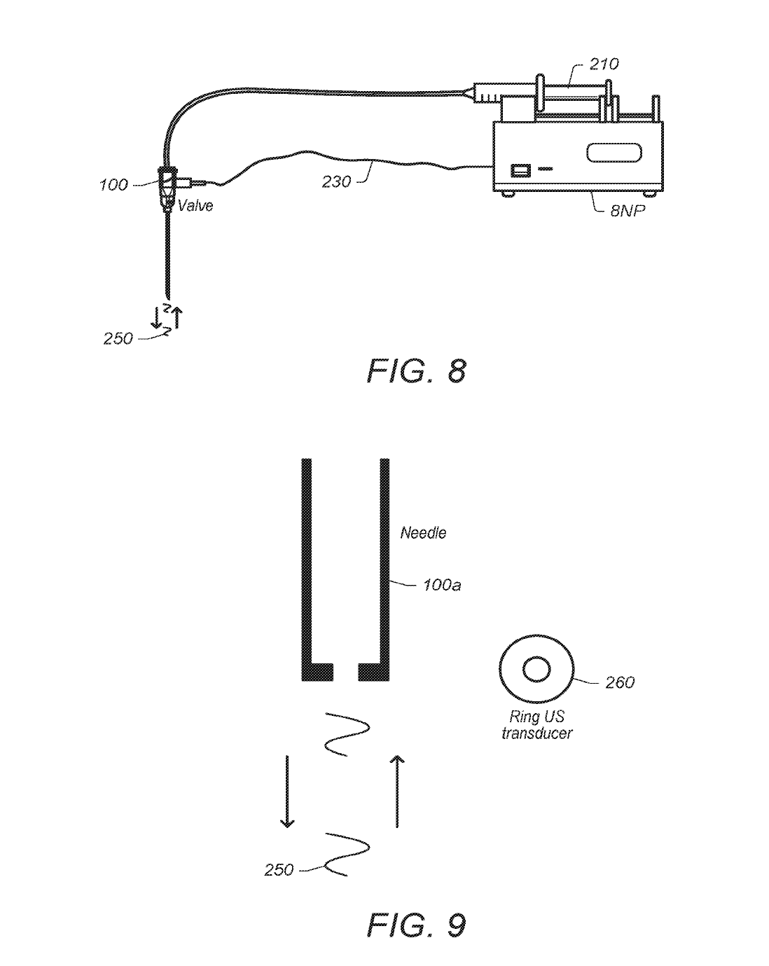

In some embodiments, the needle tip contains an ultrasonic transducer. FIGS. 7-8 depict embodiments of a representation of a smart acoustic syringe 100. In some embodiments, an ultrasound element 240 is embedded in the tip or toward the end of the needle and sends and receives ultrasonic pulses 250. Electronics for the system re contained in a separate module coupled through a separate electrical cable 230. FIG. 8 depicts an embodiment of a representation of the smart acoustic syringe 100. Electronics for the system are contained in an external infusion pump 210 coupled through a separate electrical cable 230.

The device may contain only an ultrasonic transducer or may also contain some embodiment of the electrodes described above and have multiple sensing modalities. In some embodiments, the transducer may include a piezoceramic, piezocomposite, capacitive micromachined ultrasound transducer (CMUT), Polyvinylidene fluoride (PVDF), or other material for sending acoustic signals in response to a voltage stimulus. In some embodiments, the ultrasound transducer may include a ring transducer with a hole in the center that is excited along its thickness mode. FIG. 9 depicts an embodiment of a detail of the needle tip 100a of the smart acoustic needle 100. The acoustic transducer 260 is a circular disk that is embedded in the needle tip. The disk operates at a frequency several times larger than the size of the disk so that the ultrasound energy emanating from the tip is directional. With this configuration, the drug may be injected through the center of the transducer. If a small, 0.7 mm diameter needle or similar size is used, he transducer may include a high frequency ultrasound transducer at a working frequency of 1-20 MHz (preferable 10 MHz) so that the transducer acts as a directional piston source. For example, a transducer in piezoceramic would have a thickness of 200 microns at 10 MHz if it was excited in its thickness mode. In some embodiments, while the electronics for driving the element may be separate from the needle/hub assembly, there may be a small pre-amplifier located in the needle hub that is powered by external electronics. The electronics to the element may include a pulser/receiver or other mode of sending short ultrasound waves at high frequencies (e.g., 10 MHz) and receiving them.

In some embodiments, when the device sends out short ultrasonic pulses, the ultrasound element may detect when it is in a free tissue space or facing an object that is acoustically reflective. By probing the region around the implantable pump or other device, the location of the septum may be determined. The device may be used to orient the needle tip at 90 degrees to the pump surface to avoid oblique insertion through the pump septum. For example, if the septum material allows for transmission of some of the ultrasonic wave, multiple reflections within the septum may be detected when the transducer is facing the septum material.

Alternatively, the septum may act as a perfect absorber of ultrasound energy and no reflection may appear when the needle tip is perpendicular to the septum surface. When the transducer is inserted into the septum, the received signal may diminish or disappear (and electrical impedance of the ultrasound element may change) and it will further change once the element is through the septum and inside the pump reservoir or inside the housing of the catheter. Furthermore, the transducer may be used to measure the amount of residual drug in the reservoir by measuring the time of flight of a sent pulse and knowing the sound speed of the drug.

FIG. 12 depicts an embodiment of the method of use of the smart needle system 100 with an ultrasonic transducer at the tip. The user activates the smart needle device prior to insertion in the patient (1202). Activation of the device begins stimulation of the ultrasound on the needle tip (1204) and measurements of its electrical impedance, which are processed by the microprocessor (1206). When the device correctly senses that it has been inserted through the septum and into the port, it will allow for the user to refill the device (1208). The smart needle may further communicate with the external infusion pump through RFID, direct electrical connection, or other means to notify the pump that injection of drug can proceed.

In some embodiments, the proposed device may also consist of a combination of electrical sensors/contacts and acoustic elements and/or be used in combination with other sensors.

In some embodiments, the interface may contain additional electronics including wireless electronic communication devices (e.g., RFID or Bluetooth). For example, RFID may be used to communicate with the implanted pump system. For example, Bluetooth or another means of electronic transmission can be used to communicate with an external device such as a smartphone. The smartphone may include software for displaying information from the device.

In some embodiments, the hub may contain a “lockout” mechanism that prevents the drug from exiting the needle tip if certain criteria are not defined. This “lockout” mechanism may include a microvalve near the needle tip that does not allow for injection of medications unless the microprocessor/controller validates that the needle/syringe is properly inserted into the correct port in the implanted drug delivery device. These valves may include, for example, solenoid microvalves such as those made by Takasago (http://www.takasago-fluidics.com/pdf/catalog/FVseries.pdf), or may consist of alternative designs such as those listed by Kwang et aL 2006 [A Review ofMicrovalves, J. Micromech. Microeng. 16 (2006) R13-R39] which is incorporated by reference.

In some embodiments, a security measure may exist within the hub/syringe interface.

If the smart needle hub is used with a pre-filled syringe, an anti-tamper mechanism may exist in the hub. The anti-tamper mechanism may alter the drug making the drug not useable (e.g., for recreational use). This anti-tamper mechanism may consist of a dye, an emetic drug, or other substance that is released if the user tries to tamper with the needle/hub mechanism.

In some embodiments, the proposed smart needle may include a single-use device that can be used with a normal syringe full of medication. It can also be part of a pre-filled syringe device.

In some embodiments, the device may include indicators that notify the user when the device is ready for injection of the drug. These indicators may include colored LEDs, sound, or 20 other indicator that will notify the user when the smart needle is properly inserted into the septum.

In some embodiments, the device may interact with an external controller through RFID, Bluetooth, or another form of radiofrequency communication. This information may be communicated to another device to indicate the drug information such as: drug type, manufacturer, serial number, concentration, date of re-fill. The smart needle may also communicate directly with the implanted pump system to communicate the drug information so that the pump can store this information for later retrieval. In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the of such conflict, then any such conflicting text in such incorporated by reference U.S. patents, U.S. patent applications, and other materials is specifically not incorporated by reference in this patent.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of ensuring proper transfer of a fluid to a medical device using a syringe, comprising:
inserting a distal end of a needle of a syringe into a medical device;
determining a position of a distal end of the needle relative to a fluid receiving area of the medical device using a sensor; and
determining if the position of the distal end of the needle relative to the medical device is within predefined parameters relative to the fluid receiving area.

2. The method of claim 1, further comprising:
inhibiting transfer of fluid from the syringe to the medical device if the position of the distal end of the needle relative to the medical device is not within the predefined parameters relative to the fluid receiving area.

3. The method of claim 1, further comprising:
allowing transfer of fluid from the swinge to the medical device if the position of the distal end of the needle relative to the medical device is within the predefined parameters relative to the fluid receiving area.

4. The method of claim 1, further comprising:
continuously determining, during use, if the position of the distal end of the needle relative to the medical device is within predefined parameters;
inhibiting transfer of fluid from the syringe to the medical device if the position of the distal end of the needle is within predefined parameters; and
resuming transfer of fluid from the syringe to the medical device if the position of the distal end of the needle relative to the medical device is within the predefined parameters.

5. The method of claim 1, wherein inhibiting fluid flow out of the syringe to the medical device comprises physically inhibiting movement of a plunger in the syringe.

6. The method of claim 1, wherein the medical device comprises a container, and wherein the contents of the syringe are transferrable to the container while the medical device is implemented in a patient.

7. The method of claim 1, wherein determining a position of a distal end of the needle further comprises determining electrical resistance to a current applied to at least a portion of the needle.

8. The method of claim 7, wherein the electrical resistance is determined based on electrical signals received from electrodes associated with the syringe.

9. The method of claim 8, wherein three sets of electrodes are associated with the syringe.

10. The method of claim 9, wherein a first set of electrodes is positionable in a subject's tissue, a second set of electrodes is positionable in a container of the medical device, and a third set of electrodes are positionable in a septum of the medical device.

11. The method of claim 8, wherein determining a position of a distal end of the needle further comprises determining a resistance to electrical current applied to at least a portion of the needle using at least two sets of electrodes associated with the syringe, and further comprising identifying a first substance a first set of electrodes is positioned in during use based on a first determined resistance and identifying a second substance a second set of electrodes is positioned in during use based on a second determined resistance.

12. The method of claim 1, wherein the medical device comprises an implanted device at least partially positioned in the subject and further comprising sending and/or receiving at least one packet of electronic information to and/or from the implanted device using an electronic communications system associated with the syringe.

13. The method of claim 1, further comprising transferring at least one packet of electronic information between the medical device and a control system using an electronic
communications system associated with the syringe.

14. The method of claim 1, wherein the medical device comprises an implanted device at least partially positioned in the subject and further comprising ending and/or receiving at least one packet of electronic information to and/or from the implanted device using one or more electrodes associated with the syringe.

15. The method of claim 1, wherein determining a position of a distal end of the needle further comprises emitting short ultrasonic pulses from at least a portion of the needle.

16. The method of claim 1, wherein determining a position of a distal end of the needle further comprises emitting short ultrasonic pulses from at least a portion of the needle using an ultrasonic transducer associated with the syringe.

17. The method of claim 1, further comprising:
determining a relative amount of material in a container of the medical device using an ultrasonic transducer associated with the syringe.

18. The method of claim 1, wherein the syringe comprises at least one electrode and at least one ultrasonic transducer associated with the syringe.

19. A method of ensuring proper injection of a patient using a syringe, comprising:
inserting a distal end of a needle of a syringe into a patient;
determining a position of a distal end of the needle relative to a desired injection point using an electronic sensor; and
injecting fluid from the syringe into the patient when the sensor indicates that the distal end of the needle has reached the desired injection point.

20. A syringe system, comprising:
a tubular body open at its proximal end and opening to a hollow needle at the distal end;
an electrical sensor positioned on the needle;
a signal detector for receiving and interpreting signals from the sensor.
